(19) United States
(12) Patent Application Publication
(10) Pub. No.: US 2004/0265366 A1
(43) Pub. Date: Dec. 30, 2004

(54) Metallothioneine-containing liposomes

(76) Inventors: Juan Hidalgo Pareja, Bellaterra (ES); Pilar Gonzalez Duarte, Bellaterra (ES); Pedro Gonzalez Ensenat, Barcelona (ES); Milena Penkowa, Copenhagen (DK)

Correspondence Address: STEINBERG & RASKIN, P.C., 1140 AVENUE OF THE AMERICAS, 15th FLOOR, NEW YORK, NY, 10036-5803, US

(21) Appl. No.: 10/493,615

(22) PCT Filed: Oct. 24, 2002
(86) PCT No.: PCT/IB02/04477

(30) Foreign Application Priority Data
Oct. 26, 2001 (ES) P200102435

(51) Int. Cl. A61K 9/127; A61K 38/17

(52) U.S. Cl. 424/450; 514/12

(57) ABSTRACT

Comprises a dispersion of nanocorpuscles in an aqueous solution as a dispersant and a pharmaceutically acceptable alcohol as co-dispersant or cosolvent, said nanocorpuscles having at least one lipid bilayer comprising at least one biliar salt and at least one phospholipid with the general formula (I), in which R₁ and R₂ are the same as each other or different, being generally linear aliphatic residues with 12-22 carbon atoms, having up to 4 double cis bonds, preferably up to 3 double cis bonds; these nanocorpuscles having an average diameter lower than 500 nm; and including metallothioneine in their interior.

The process for obtaining comprises: a) prepare an aqueous solution of at least one biliar salt and metallothioneine; b) prepare an alcoholic solution of the phospholipid with the general formula (I); c) jointly homogenise both solutions.

This pharmaceutical composition can be used as a medicament, particularly in the treatment of neurodegenerative and neurological disorders.

### FIELD OF THE INVENTION

The present invention refers to a pharmaceutical composition of a dispersion of lipid vesicles containing metallothioneine, and which can be used in the manufacture of a medicament for the treatment of disorders in which metal sequestration, the reduction of oxidative stress and apoptosis or an increase in cell survival are, inter alia, a principal or accessory part of therapy.

The present invention also refers to the process for obtaining said pharmaceutical composition.

### BACKGROUND TO THE INVENTION

Metallothioneines (MTs) form a family of proteins with a low molecular weight (6-7 kDa) which owe their name to the fact that they possess an unusual abundance of cysteine residues and the fact that they have the capacity to bind heavy metals, such as Zn(II) and Cu(I). These proteins are usually isolated from tissue as zinc-MT.

In accordance with their structural characteristics, MTs are subdivided into different families. Mammalian MTs are made up of four main isoforms, known as MT-1 to MT-4. MT-1 and MT-2 are expressed in the majority of tissues, including the brain, while MT-3, also known as growth inhibitory factor (GIF) due to its effect on rat neurons in primary culture, is expressed predominantly in the central nervous system. MT-4 is basically expressed in the stratified squamous epithelium.

All MT isoforms have been related to different physiological functions, such as the metabolism of zinc and copper, protection against reactive species of oxygen or adaptation to stress, inter alia. In the case of MT-3, its involvement has been suggested in neuromodulation processes and in the pathogenesis of Alzheimer's disease.

Furthermore, there are clear indications that the MT-1 and MT-2 isoforms could be important protective factors for the central nervous system. Some researches indicate that MT-1 and MT-2 are found to be increased in certain human neurodegenerative disorders, such as Alzheimer's disease, Pick's disease and amyotrophic lateral sclerosis. In addition, it has been observed that a significant increase in MT-1 and MT-2 occurs following a brain lesion caused by stress, cryolesion, seizures induced by kainic acid, NMDA, 6-aminonicotinamide and ischemia, as well as by the expression of proinflammatory cytokines in transgenic animals. It has also been observed that the over-expression of MT-1 in transgenic mice protects against mild focal cerebral ischemia and reperfusion, while mice lacking MT-1 and MT-2 show an altered inflammatory response, an increase in oxidative stress and apoptosis and a retarded capacity of healing wounds after a focal cryolesion.

Therefore, use of metallothioneine in the treatment of these pathologies seems to be a viable alternative. However, the use of metallothioneine on its own does not allow a proper treatment of all these pathologies because said protein is not capable of passing through the blood-brain barrier. Thus, there is not yet a viable and effective solution in the state of the art that allows to said protein to pass through the said barrier.

In this regard, liposomes represent a good alternative since, given their size and their physical and chemical characteristics, these structures circulate, penetrate and spread through tissue with great efficacy, carrying, in their interior, the active principle. The characteristics and properties of a liposomal formulation are determined by its composition, method of preparation, active principle, etc., and the different components must be adjusted in order to achieve the desired result.

During last years, several compositions made up of lipid vesicles comprising an active principle in their interior have been described.

WO93/18750 describes unilamellar liposomes comprising phospholipids and including an antidote, in order to avoid damaging certain cells when administering a drug that does not possess a cellular action that is focussed solely on the cells or microorganisms to be treated.

DE2730570 describes an injectable solution comprising a lipid, a benzodiazepine, an additive to make the solution isotonic with the blood and tissular fluid and a cholanic acid derivative with the general formula (I):

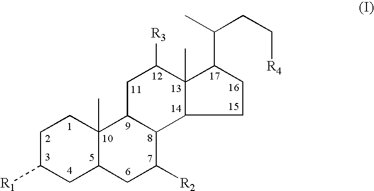

In the aforementioned document, the addition of lipids to the injectable solution allows the reduction or elimination of disadvantages such as haemolytic activity, caused when natural micelle formers are used.

DE4341479 describes the use of metallothioneines and apothioneines contained in liposomes, for the treatment of viral infections.

However, none of said documents describes or suggests a liposomal formulation with improved characteristics, such as the capacity to penetrate through tissue, the capacity to adapt, both chemically and physically, to different environments, etc.

The authors of this invention have discovered a pharmaceutical composition formed of lipid vesicles containing metallothioneine which can improve the pharmacokinetic and pharmacodynamic behaviour of metallothioneines administered on their own.

The present invention also refers to the use of said composition in the manufacture of a medicament for the treatment of inflammatory, degenerative and intoxication processes or the accumulation of heavy metals and, in particular, neurological illnesses in which there is a need to sequester metals, reduce oxidative stress and apoptosis or increase cell survival.

### DESCRIPTION OF THE INVENTION

The present invention refers to a pharmaceutical composition comprising a dispersion of nanocorpuscles in an aqueous solution as a dispersant and a pharmaceutically acceptable alcohol as co-dispersant or cosolvent, said nanocorpuscles having at least one lipid bilayer comprising at least one biliar salt and at least one phospholipid with the general formula (I):

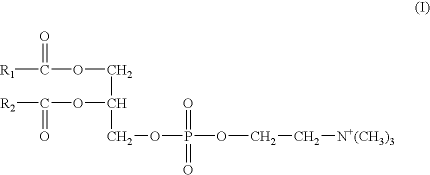

in which R₁ and R₂ are the same as each other or different, being generally linear aliphatic residues with 12-22 carbon atoms, having up to 4 double cis bonds, preferably up to 3 double cis bonds;

these nanocorpuscles having an average diameter lower than 500 nm; and including metallothioneine in their interior.

The average diameter of each nanocorpuscle is preferably lower than 250 nm. In this way, the diffusion of the pharmaceutical composition through tissue is improved.

In this invention, the term “nanocorpuscles” is understood as nanoparticles, micellar elements, substances that can be converted into micelles (reversible), liposomes (uni-, oligo- or multimellar) and nanocolloids, with an average diameter lower than 500 nm, preferably lower than 250 nm, with standard deviations in the order of 30%.

In a preferred embodiment of the present invention, metallothioneine is found in a concentration of 1 mg to 100 mg per litre of prepared composition.

In the present invention, the term “metallothioneine” is understood as a metallothioneine formed by one or more of the isoforms and/or sub-isoforms of the protein, both native and recombinant, with the entire or partial amino acid sequence, in a normal or mutated sequence, as well as the natural, synthetic or semi-synthetic derivatives and mixtures thereof.

The metallothioneine proteins comprised in the nanocorpuscles can have a different metal content. Likewise, these proteins can be isolated from any organism.

In the present invention, the term “dispersant” means an aqueous solution comprising water, although the addition of electrolytes such as salts (e.g. NaCl), buffers, etc. is not excluded.

In this invention, the dispersant in the composition is preferably water.

In addition, the biliar salt or salts are selected from the group constituted by sodium cholate, sodium desoxycholate, sodium glycocholate, sodium taurocholate, sodium taurodeoxycholate, sodium ursocholate and sodium quenoxycholate, or their derivatives.

In a preferred embodiment of the invention, the pharmaceutically acceptable alcohol is preferably ethanol.

Stable and/or resistant liposomal formulations as well as flexible liposomal formulations are known in the state of the art, this being due both to their composition and to the technique used in their manufacture. In this invention, the combined presence of both a biliar salt and a cosolvent or co-dispersant (together with the phospholipid which is the structural base of the liposome and is capable of forming structures by itself) results in a balanced combination of stability (greater resistance) and flexibility (greater molecular movement), a fact which gives to this particle such advantageous characteristics, among others, withstanding (adapting to) osmotic, ionic and polarity changes in the medium.

As a consequence of the characteristics mentioned above, the nanocorpuscles of the pharmaceutical composition in this invention (which carry the active principle incorporated, in this case metallothioneine) circulate and spread more easily through the different structures, tissues and organs, e.g. the blood-brain barrier and the central nervous system, thus improving certain aspects of the pharmacokinetic and pharmacodynamic behaviour of metallothioneine on its own.

In addition, the alcohol and the biliar salts offer advantages in the production of the liposomal formulation, either, for example, by facilitating the dispersion of the phospholipids (due to the presence of the alcohol), or by reducing the amount of energy necessary for its production, allowing the labile products to be made in capsule form (due to the presence of the biliar salt).

Advantageously, in this invention, the proportion of alcohol to phospholipid is between 0.5/1 and 3/1, being, preferably, said proportion 1/1 in volume/weight.

In a preferable aspect of the present invention, the phospholipid is soy lecitine.

Advantageously, the concentration of phospholipid in the composition of the invention is between 0.03 g and 200 g per litre of composition, said concentration, preferably, being, between 1 g and 100 g per litre of composition.

In a preferred embodiment of this invention, the molar ratio between phospholipid and biliar salt ranges between 2 and 10, more preferably between 3 and 5.

In another preferred embodiment of the present invention, the pharmaceutical composition may include, at least, one additive, one conventional coadjuvant and/or its mixtures, examples of which are agents for providing isotonicity, antioxidants, buffers, tensioactive or cotensioactive agents such as polysorbates or free fatty acids, thickening agents, preservatives, etc.

Surprisingly, the composition of the present invention shows a great capacity for the diffusion of the vesicles through tissues and a great ease in passing through barriers (such as, for example, the blood-brain barrier), meaning that it can be used for the manufacture of a medicament for the treatment of neurodegenerative and neurological disorders, such as Alzheimer's disease, Parkinson's disease, multiple sclerosis, amyotrophic lateral sclerosis, viral and bacterial meningitis, spongiform encephalopathy and AIDS encephalopathy.

In addition, the composition in the present invention can be used for the manufacture of a medicament for the treatment of any neurological pathology in which there is an associated inflammatory response and the production of free radicals (oxidative stress), as well as damage caused following traumatic injury.

Furthermore, this composition could be used to manufacture a medicament for the treatment of any neurological disorders in which there is an accumulation of metals such as copper, Wilson's disease or Menkes disease, or in cases of poisoning by metals such as Cu, Cd, Zn, Pb, etc.

The composition of the present invention can also be used for the manufacture of a medicament for the treatment of diseases of peripheral tissues in which the main or accessory therapy requires the scavenging of metals, the reduction of oxidative stress and apoptosis, or an increase in cell survival, such as, for example, diseases in which an inflammatory response is produced, such as arthritis, as well as oxidative stress, such as reperfusion injuries, post-traumatic injuries or metal accumulation.

In addition, the present invention also refers to a process for obtaining a composition in accordance with the definition set out above, comprising the following stages:

a) Prepare an aqueous solution of at least one biliar salt and metallothioneine;

b) Prepare an alcoholic solution of the phospholipid with the general formula (I);

c) Jointly homogenise both solutions.

By way of an advantage, in the process of the present invention, after each of these stages and/or at the end of the process, one can carry out an additional dilution stage, adding at least one additive, a coadjuvant, a pharmaceutically acceptable excipient and/or mixtures thereof.

Thus, with the process of the present invention one can obtain a composition with each of the components (phospholipid, biliar salt or salts, etc.) at a concentration level that is ideal for its administration, or one can obtain a composition that requires an additional subsequent dilution stage (adding at least one additive, a coadjuvant, a pharmaceutically acceptable excipient and/or mixtures thereof), in such a way that a final pharmaceutical composition that is ideal for administration is provided.

The dilution stage is preferably carried out with the addition of water.

In a preferred embodiment of the process of the invention, said process comprises at least one filtration stage after each of the process stages and/or at the end of the process, said filtration stage being, preferably, one of sterile filtration.

The composition of the present invention can be administered parenterally, orally, intravenously, etc., and, more generally, one may use any of the galenic forms that do not, by their particular nature or by their components, impede or improperly reduce the release of the metallothioneine in the area to be treated.

Pharmacological Properties.

On giving the composition of the present invention to mice, none of the classic signs of toxicity, such as loss of weight or reduction in food intake, were observed. The composition was significantly protective in a brain damage model (cryolesion of the cortex), restricting inflammatory response, reducing oxidative stress and drastically decreasing cell-death rate (mostly neuronal), including the cell death caused by apoptosis. As a consequence, damage was lower and regeneration of the affected area was much greater.

It is interesting that the composition of the invention has also been effective in carrying metallothioneine through the blood-brain barrier, since in undamaged animals, with the barrier intact, we have been able to detect metallothioneine by immunohistochemistry in the central nervous system. This does not occur if one injects metallothioneine on its own (without it being encapsulated in liposomes).

These results show the great capacity for diffusion that the composition of the invention has in the tissues, meaning that the composition can be used for the manufacture of a medicament for the treatment of brain pathologies in which the blood-brain barrier has not been altered.

At the same time, this composition can be used in the manufacture of a medicament for the treatment of pathologies of peripheral tissues, in which it will have effects and advantages similar to those observed in the brain, i.e. anti-inflammatory, anti-oxidant and anti-apoptotic effects. It would also be highly effective in sequestering heavy metals such as Cd, Cu, Hg, Pb and others, as well as Zn.

The following examples are provided by way of illustration, and they in no way restrict the invention.

### FIGURES

FIG. 1: it is a graphic wherein it is shown a rotational correlation time (τ) variation when the ethanol ratio in the medium is increased, as it is described in Example 1.

### EXAMPLES

Other characteristics and advantages of the invention will become clearer from the additional description that follows, which is meant purely by way of example and is in no way restrictive. This additional description will be given with reference to the forms of the invention that are currently preferred.

### Example 1

### Study of the Liposome's Adaptation to Environments of Differing Polarity

The aim of the following example is to assess the effect of the presence of the cosolvent on the mobility of the phospholipids forming the liposome.

1.A. Formation and Characterization of Liposomes.

The liposomes are formed using the standard evaporation-hydration process in an aqueous medium with subsequent extrusion.

Once formed, a gradual change is made to the polarity of the medium with the addition of different volumes of ethanol, finally achieving ethanol proportions of: 10%, 15%, 20%, 25%, 30%, 40% and 50%, with the aim of inducing the inversion of the lipids from the external monolayer, thus forming what we will call the inverse liposomes. The final lipid concentration was 10 mg/ml.

In order to quantify the level of inversion of the lipids in the external monolayer due to the effect of the change in the medium's polarity, electronic paramagnetic resonance (EPR) was used. This technique is capable of monitoring the molecular dynamics of many substances. Given that the lipids forming the liposomes lack paramagnetic molecules, in order to study the potential inversion of the lipids in the external monolayer a probe was included in the liposomes' lipid stage: the radical spin marker 16-doxyl stearic acid.

The EPR spectra made it possible to calculate the τ parameter, the rotational correlation time. This parameter denotes the degree of freedom of electronic spin movement, its value being inversely related to the mobility of the probe introduced into the bilayer, calculated using the following equation:

τ=6.5×10⁻¹⁰W₀[(W₋₁/W₀)−1]

in which W₀ y W₋₁ represent the width of the lines in the medium and high fields of the first derivative of each absorption spectrum.

DLS experiments showed that the phospholipid liposomes in a pure aqueous solution were communities of stable, spherical and unilamellar vesicles, with an approximate diameter of 150 nm and a polydispersity index of 0.01

Both EPR and DLS techniques showed that proportions of ethanol exceeding 30% in respect of water caused structural changes in the liposomes which were related to the beginning of the solubilisation of the vesicles, and it is considered that a proportion of 30% ethanol is the admissible in order to guarantee the liposome's integrity.

TABLE
Variation in the size and polydispersity index of liposomes
when increasing the proportion of ethanol in the medium.

| | | POLYDISPERSITY |
|---|---|---|
| % ETHANOL | DIAMETER (nm) | INDEX |
| 0 | 150 | 0.010 |
| 10 | 170 | 0.020 |
| 20 | 180 | 0.050 |
| 30 | 200 | 0.100 |
| 40 | 600 | 0.578 |
| 50 | 1500 | 0.899 |

1.B. Study of the effect of the Polarity of the medium on the Mobility of the Lipids in the Liposomes' External Monolayer.

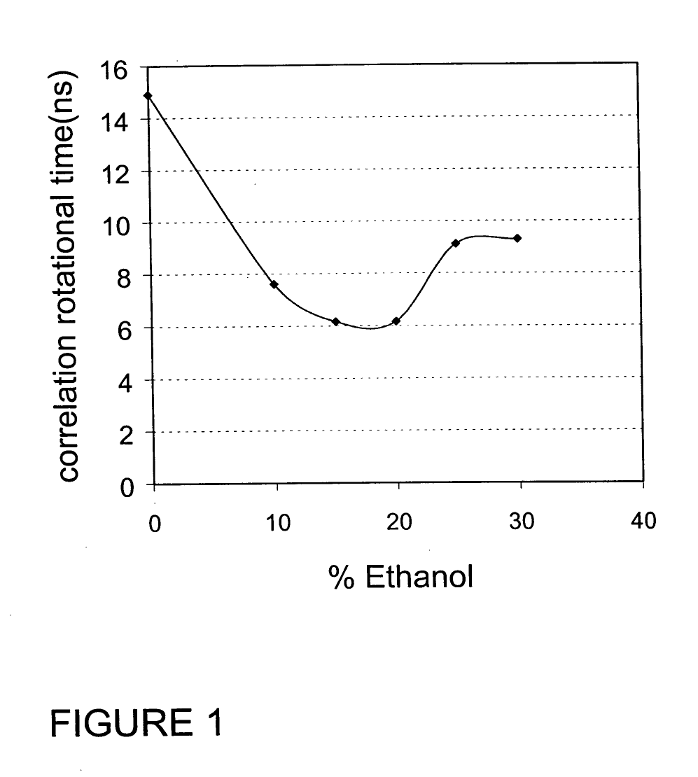

The τ parameter (rotational correlation time) is inversely related to the mobility of the probe, and therefore the mobility of the lipids in the bilayer. Thus, a reduction in this parameter when the proportion of in the medium is increased indicates an increase in molecular mobility, as shown in FIG. 1.

The most interesting results were seen in solutions containing between 10 and 20% ethanol approximately. With these proportions, maximum molecular mobility was obtained ithout any change in the physical and chemical parameters of the liposomes (lamellarity, size and shape).

At between 20% and 30% ethanol, an increase in τ was observed, in mother words, a reduction in the mobility of the lipids that could be attributed to the fact that the number of inverted lipid molecules has increased, thus obstructing the mobility of the probe. Proportions of ethanol in excess of 30% caused the destabilisation of the liposomes, a result which confirmed the EPR and DLS findings.

It was confirmed that liposomes in media containing less than 30% ethanol remained stable for at least a week.

Thus, EPR technique showed that the ethanol was capable of inducing molecular mobility in the lipids forming the liposomes, and this mobility was related to the process of the inversion of the lipids forming the liposomes' external monolayer, creating inverse liposomes and being able to cause an increase in the flexibility of the bilayer, which could result in the deformation of the liposomes.

As a consequence, the results obtained indicate a spontaneous inversion of the lipids in the liposomes' external monolayer, depending on the polarity of the medium. This could be considered to be an adaptation of the liposomes in explaining their passage through the different structures and tissues of the central nervous system and other structures, tissues and organs.

By way of conclusion, the presence of ethanol in the pharmaceutical composition in this invention offers molecular mobility to the liposomes, which in turn provides a capacity to adapt to the polarity of the medium, a fact which explains the passage of the liposomes through the different barriers and tissues with a particular polarity.

### Example 2

### Obtaining of an Encapsulated Eetallothioneine Reparation in Liposomes

2.1. Preparation and Characterisation of Metallothioneine (MT).

A solution with a known concentration of MT protein is prepared (with the commercial brand Sigma) in a controlled atmosphere of inert gas (Ar or N₂), the pH of the solution is adjusted to the desired value with a suitable buffer solution (pH 7-8 for holo-MT and a pH of less than 3 for apo-MT), and its S, Zn and Cd content is checked using ICP-AES, along with the level of protein oxidation (Ellman method).

The relevant form, holo-MT or apo-MT, is characterised by UV visible spectroscopy, circular dichroism (CD) and electro-spray ionisation mass spectroscopy (ESI MS).

In addition, in the case of obtaining holo-MT, the molecular weight of the apo-MT form is determined by acidifying a fraction of the earlier solution and recording its mass spectrum by ESI MS.

2.2. Encapsulating MT in Liposomes

a) 30 g of lecitine are dissolved in 30 ml of ethanol at 96%.

b) 4.40 g of sodium cholate are dissolved in 220 ml of water. To this solution, 20 ml of an aqueous solution containing 1 mg of MT/ml is added.

The solutions obtained in a) and b) are mixed in a homogenizer and are then subjected to sterile filtration or filtration with simultaneous sterilisation, giving a fine dispersion which is diluted to the desired concentration for administration. Preliminary dilution or, directly, the final dilution can be carried out before sterile filtration, in order to facilitate this process.

2.3. Determination of the amount of MT Encapsulated in the Liposome

In order to determine the amount of MT, either in apo-MT or holo-MT form, that is found in the lipid phase of the liposome, a small amount of the homogenised MT solution encapsulated in the liposomes is taken, and the two phases, aqueous and lipid, are separated by ultracentrifuge using suitable filters, in accordance with the molecular weight of the protein (MWCO). A fraction of each of the separated stages is analysed and their content is determined using ICP-AES (S, Zn and Cd).

### Example 3

### Effect of Administering Encapsulated Metallothioneine (MT)

Various groups of mice were treated with encapsulated MT, obtained according to the process described in the preceding example, and they were given a daily dose of 15 μg/mouse (weight=22-24 g per mouse), for 3 or 7 days, subcutaneously, both to control mice and to mice that had been subjected to a cryolesion of the cortex. The cryolesion was made by anaesthetising the mice with ketamine/xylazine (100/10 mg/kg IP), then subsequently opening up the skin with a scalpel and applying a small rod of dry ice to the skull for 60 seconds; immediately afterwards, povidone iodide was applied and the cut sutured. The animals were killed at different times with an anaesthetic dose of 100 mg/kg Brietal (Methohexital 10 mg/ml, Eli Lilly) after the perfusion of the heart with 4% paraformaldehyde in order to fix the brain and other organs.

The response of the area surrounding the injured cortex was analysed, with an examination of astrocyte reactivity (GFAP immunostaining), the recruitment and activation of macrophages (lectin staining), the recruitment of T lymphocytes (CD3 immunostaining), oxidative stress (immunostaining for nitrosylated proteins in Tyr residues, and for malondialdehyde) and apoptosis (TUNEL technique). We also analysed MT levels using immunohistochemistry.

Astrocytosis was significantly increased by the composition. In addition, the recruitment of macrophages and T lymphocytes in the injured area was drastically reduced by treatment with our liposome-MT composition (down to 20-30% of the mice treated only with the empty liposome-without MT). A similar reduction was observed in oxidative damage and apoptosis of the cortex surrounding the lesion.

These results show the powerful inhibiting effect of liposome-MT on inflammatory response in the brain, and a clear protective effect in reducing oxidative damage and cell death (mainly neuronal, but also affecting other cell types). In the same way, it has been demonstrated that MT administered as liposome-MT effectively reaches the extracellular space in the brain, since increased levels of MT were detected not only in the cryolesion area (as was expected, as the normal cytoarchitecture had been broken and the blood-brain barrier opened with the massive breakage of blood vessels, etc.) but also in many other areas of the brain with their barrier intact, something that was not observed in animals injected with MT without liposomes.

Although the invention has been described specifically in terms of the forms of execution considered at present, those with knowledge in this field will understand that this is not intended to be restrictive, since the invention may only be considered to be limited by the terms of the following claims, understood in their broadest sense, and covering the technical equivalents of the elements and provisions specified therein.

1. A pharmaceutical composition comprising, a dispersion of nanocorpuscles in an aqueous solution as a dispersant and a pharmaceutically acceptable alcohol as co-dispersant or cosolvent, said nanocorpuscles having at least one lipid bilayer comprising at least one biliar salt and at least one phospholipid with the general formula (I): 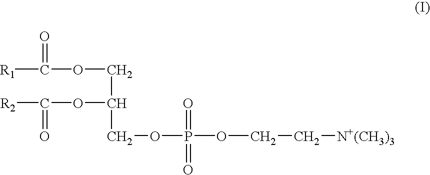
in which R₁ and R₂ are the same as each other or different, being generally linear aliphatic residues with 12-22 carbon atoms, having up to 4 double cis bonds, preferably up to 3 double cis bonds;
these nanocorpuscles having an average diameter lower than 500 nm; and including metallothioneine in their interior.

2. A pharmaceutical composition according to claim 1, wherein each corpuscle has an average size lower than 250 nm.

3. A pharmaceutical composition according to claim 1, wherein the metallothioneine is found in a concentration of between 1 mg and 100 mg per litre of prepared composition.

4. A pharmaceutical composition according to claim 1, wherein the dispersant is water.

5. A pharmaceutical composition according to claim 1, wherein the co-dispersant or cosolvent is ethanol.

6. A pharmaceutical composition according to claim 1, wherein the biliar salt is selected from the group constituted by sodium cholate, sodium desoxycholate, sodium glycocholate, sodium taurocholate, sodium taurodeoxycholate, sodium ursocholate and sodium quenoxycholate, or their derivatives.

7. A pharmaceutical composition according to claim 1, wherein the alcohol/phospholipid proportion ranges between 0.5/1 and 3/1 in volume/weight.

8. A pharmaceutical composition according to claim 7, wherein said alcohol/phospholipid proportion is 1/1 in volume/weight.

9. A pharmaceutical composition according to claim 1, wherein the phospholipid is soy lecitine.

10. A pharmaceutical composition according to claim 1, wherein the phospholipid concentration is between 0.03 g and 200 g per litre of composition.

11. A pharmaceutical composition according to claim 10, wherein this phospholipid concentration is between 1 g and 100 g per litre of composition.

12. A pharmaceutical composition according to claim 1, wherein the molar ratio between phospholipid and biliar salt is between 2 and 10.

13. A pharmaceutical composition according to claim 12, wherein said molar ratio between phospholipid and biliar salt is between 3 and 5.

14. A composition according to claim 1 wherein it includes at least one additive, one coadjuvant and/or their mixtures.

15. Process for obtaining a pharmaceutical composition in accordance with claim 1 wherein it includes the following stages:
a) Prepare an aqueous solution of at least one biliar salt and metallothioneine.
b) Prepare an alcoholic solution of the phospholipid with the general formula (I);
c) Jointly homogenise both solutions.

16. Process according to claim 15, wherein it includes at least one filtration stage after each of the process stages and/or at the end of the process.

17. Process according to claim 16, wherein said filtration stage is one of sterile filtration.

18. Process according to claim 15, wherein after each of the stages and/or at the end of the process, it includes a dilution stage.

19. Process according to claim 18, wherein said dilution stage is carried out with the addition of at least one additive, a coadjuvant, a pharmaceutically acceptable excipient and/or mixtures thereof.

20. Process according to claim 19, wherein said dilution stage is carried out with the addition of water.

21. A pharmaceutical composition comprising a dispersion of nanocorpuscles in an aqueous solution as a dispersant, according to claim 1, for use as a medicament.

22. Use of a pharmaceutical composition according to claim 1 for the manufacture of a medicament for the treatment of neurodegenerative and neurological disorders such as Alzheimer's disease, Parkinson's disease, multiple sclerosis, amyotrophic lateral sclerosis, viral and bacterial meningitis, spongiform encephalopathy and AIDS encephalopathy.

23. Use of a pharmaceutical composition according to claim 1 for the manufacture of a medicament for the treatment of neurological pathologies in which there is an associated inflammatory response and the production of free radicals (oxidative stress), as well as damage caused following traumatic accident.

24. Use of a pharmaceutical composition according to claim 1 for the manufacture of a medicament for the treatment of neurological illnesses in which there is an accumulation of metals like copper, or in cases of metal poisoning.

25. Use of a pharmaceutical composition according to claim 1 for the manufacture of a medicament for the treatment of non-neurological illnesses in which metal sequestration, the reduction of oxidative stress and apoptosis or an increase in cell survival are a principal or accessory part of therapy.
